(19) United States
(12) Patent Application Publication
(10) Pub. No.: US 2004/0161852 A1
(43) Pub. Date: Aug. 19, 2004

(54) Method of assaying an Fe-Sugar complex

(76) Inventors: Christopher G. Anderson, Salt Lake City, UT (US); Blaine T. Messenger, Sandy, UT (US)

Correspondence Address: JONES DAY, 222 EAST 41ST ST, NEW YORK, NY, 10017, US

(21) Appl. No.: 10/368,057

(22) Filed: Feb. 14, 2003

(51) Int. Cl. G01N 33/15

(52) U.S. Cl. 436/76

(57) ABSTRACT

A method for assaying a pharmaceutical product containing a Fe-Sugar complex is described herein. The assay comprises in one embodiment measurement of the molecular weight of the Fe-Sugar complex. The method described herein is robust, reproducible and can serve as a quality assurance method for pharmaceutical purposes.

### BACKGROUND OF THE INVENTION

FERRLECIT® is a stable sodium ferric gluconate complex in sucrose that is used to replete the total body content of iron. The presumed chemical structure of the FERRLECIT® complex is illustrated below.

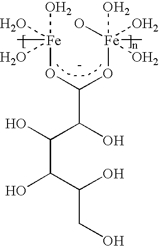

FERRLECIT® is most often administered to patients undergoing hemodialysis with and without epoetin therapy, which increases red blood cell production and iron utilization.

The current method for assaying the molecular weight of Fe-sugar complexes is aqueous gel permeation chromatography (“GPC”). The GPC method uses polysaccharide standards to construct a standard curve relating the log of the molecular weight to retention volume, when samples are passed through a column containing porous packing material and components detected by a differential refractometer. The method actually measures the relative size of the Fe-sugar complex in comparison to that of a polysaccharide standard with the same retention volume. The underlying assumption is that the Fe-sugar complex and the polysaccharide standard behave similarly under the assay conditions. As a result an average molecular weight based on the standard is calculated rather than an absolute molecular weight for the Fe-sugar complex. The GPC method yields calculated molecular weights for the FERRLECIT® complex between 289,000 and 440,000 daltons.

However, it has been observed that this methodology can be influenced by stationary phase interactions. For example, it has been discovered that one or more of the components in the FERRLECIT® complex does indeed interact by mechanisms other than size exclusion with a variety of different stationary phases. It is therefore very likely that other iron containing products such as INFeD® and VENOFER® would similarly interact with the stationary phase. These interactions can influence the outcome of molecular weight determinations made using traditional GPC.

### SUMMARY OF THE INVENTION

The invention described herein provides a valid assay method which is successful in minimizing and/or possibly quantifying the stationary phase interactions that plague the current assay method. In some aspects, the interactions, to the extent present, are reproducible. This reproducibility permits the interactions to be taken into account when calculating molecular weight of the Fe-sugar complex. The present invention does not employ a standard to measure the desired quantity.

One embodiment of the present invention employs a multi-angle light scattering (“MALS”) detector interfaced with a differential refractommetric (“DRI”) detector, both of which operate at a wavelength ranging from about 600 to about 800 nm, preferably of about 690 nm. In addition to molecular weight, size (radius of gyration also referred to as rms radius) can also be determined directly without depending upon reference-based calibration or physical assumptions about the data. Thus, the present invention permits calculation of true molecular weights based on fundamental equations rather than assumptions. Also, because the results are less dependent on interactions of the solute(s) with the stationary phase, this approach gives a much-improved determination of the molecular weight of the Fe-sugar complex (such as the sodium ferric gluconate complex in sucrose found in FERRLECIT®) compared to that obtained using the current traditional GPC procedure.

A further embodiment of the present invention utilizes a polyhydroxymethacrylate (Shodex SB 806M ) column in series with a Shodex SB guard column coupled to Wyatt Technology's Optilab DRI and DAWN EOS MALS detectors. The mobile phase comprises an alkali nitrate such as sodium nitrate, optionally containing a biocide or an antimicrobial, such as sodium azide.

### BRIEF DESCRIPTION OF THE DRAWINGS

FIG. 1. MALS (Top) and DRI (Bottom) Chromatograms Showing Benzyl Alcohol Eluting at ˜73 Minutes

FIG. 2. MALS (Top) and DRI (Bottom) Chromatograms of a Typical FERRLECIT® Analysis

FIG. 3. Reproducibility Study I, Injections 1-10

FIG. 4. Reproducibility Study I, Injections 11-20

FIG. 5. Molecular Weight Distribution vs. Injection Number For Reproducibility Study I

FIG. 6. Grouped Reproducibility Study, Group 4 (TM1512-132 4^{th} Group of 8 Injections)

FIG. 7. Reproducibility Study II, Injections 41-50

FIG. 8. Molecular Weight Distribution vs. Injection Number for Reproducibility Study II

FIG. 9. Linearity of Response

### DESCRIPTION OF THE PREFERRED EMBODIMENTS

The following terms are herein defined:

“Sugar” as used herein means glucose, sucrose, sodium gluconate and oligomers or polymers thereof or a combination thereof.

“Fe-Sugar complex” as used herein means complex comprising sugar and an iron ion that may be present in a trivalent state. The term “complex” refers to molecular and/or ionic associations governed by covalent or noncovalent interactions. Noncovalent interactions include but are not limited to, ionic, organometallic, coordinate covalent, van der Waal's hydrophobic, hydrogen bonding, dipole-dipole interactions.

The term “suitably prepared sample” refers to a sample of the pharmaceutical composition being subjected to the assay methods disclosed herein that may, in some aspects, require some preparatory work, such as dilution with mobile phase (or eluent), extraction with suitable solvents first, followed by dilution with a mobile phase, suitable derivatization before or after extraction and/or dilution, etc. Suitable sample preparation methods are standard in the art and one of skill would readily appreciate such methods.

The present invention provides a robust/reproducible assay method for determining the molecular weight of an Fe-sugar complex such as is found in pharmaceutical products useful for treating patients in need of iron therapy. The assay method is useful for determining the molecular weight of the Fe-sugar complex in pharmaceutical products such as, but not limited to, FERRLECIT®, INFeD® and VENOFER®. The assay method is valuable for validation and quality assurance purposes where a pharmaceutical manufacturer accepts or rejects certain batches and lots based on certain predetermined specifications. The specifications may be set for one or more parameters measured using the present invention, for example, Mw, Mn, Mz, Pd or a combination thereof. In one aspect, for example, the specification may be set for Mw of about 800,000±120,000, with an RSD of about 0.1% to about 0.3%. If one assayed batch results in an Mw of, for example, 400,000, the batch may be rejected as being out-of-spec and will not be distributed into the stream of commerce. This method may be distinguishable from methods that are of preparatory scale for purification or separation and purification purposes.

An assay method to determine molecular weight of an Fe-sugar complex of the present invention, employs a MALS detector (MALS DAWN EOS detector from Wyatt Technology, Santa Barbara, Calif.) interfaced with a DRI detector (Optilab DRI from Wyatt Technology, Santa Barbara, Calif.), both of which operate at a wavelength of about 690 nm.

For molecular weight determination, it is important to be able to calculate instantaneous concentration and obtain a value for the specific refractive increment, dn/dc, of the Fe-sugar complex in comparison to that of the solvent in which the analysis is conducted. This can be accomplished by coupling a DRI detector with the light scattering detector. Several methods exist for determining the dn/dc and these are known to those skilled in the art. Such techniques include manual as well as computer-assisted techniques. See for example: S. Podzimek, J. App. Polymer Sci., Vol. 54, 91-103 (1994); and P. J. Wyatt, Analytica Chimica Acta., Vol. 272, 1-40 (1993), which are incorporated herein by reference. Of the methods available for determining dn/dc, the present invention uses an “Online 100% Recovery Method.” Since the iron content of the product in question is generally known (for example, for FERRLECIT®, it is 62.5 mg of elemental iron/5 ml), the concentration in grams per milliter of the iron complex (in case of FERRLECIT®, it is presume to be sodium ferric gluconate complex) based upon the molecular weight for the currently accepted structure for the complex can be calculated. Assuming that the peak corresponding to the Fe-sugar complex represents 100% recovery, a value for specific refractive increment (dn/dc) cab be calculated using standard techniques known in the art. Such techniques may be manual as well as computer-assisted. Using a dn/dc value of about 0.08 ml/g the molecular weight is calculated to be approximately 790,000±15,000.

In summary, the present methods provide reproducible, robust and rugged assays to analyze for validation purposes of an injectable iron-containing pharmaceutical composition.

The assay methods of the present invention are illustrated by the following examples, which in no way are meant to limit the scope of the claims of the present invention but merely serve to fully define the present invention. The following supplies and conditions are used in the examples.

Equipment:

Analytical balance (0.1 mg)

Assorted pipettes, graduated cylinders and volumetric flasks

Columns: Shodex SB guard column+Shodex SB 806M @ 35° C.

DRI detector: Optilab from Wyatt Technology @ 690 nm operated at 35° C.

HPLC system: Waters Alliance HPLC System

MALS detector: DAWN EOS by Wyat Technology @ 690 nm operated at 35° C.

Microfiltration system (0.45 μm) for solvent filtration with associated vacuum source

Chemicals:

Acetic acid

EDTA sodium salt

FERRLECIT®

Milli-Q water

Sodium azide, reagent grade

Sodium nitrate, reagent grade

HPLC Conditions:

Mobile phase: 100 mM NaNO₃ plus 200 ppm sodium azide (pre-filtered through

0.45 μm nylon Millipore filters)

Flow rate: 0.5 ml/min

Sample: 1.00 ml FERRLECIT® diluted to 10.0 ml in mobile phase

Injection volume: 100 μL

Processing Method:

Calculated dn/dc=0.08 ml/g based on Online 100% Recovery Method

Fit method/model=Debye

The last injection is processed first by setting baselines for the light scattering angles and the DRI, overlaying the two curves, and defining the peak region. The peak region encompasses the entire area under both the MALS and DRI curves. The data are processed using any angles or combination of angles 4 through 18 (angles 2 and 3 are excluded due to high baseline noise from aqueous solvent). After data from the last injection are processed, the same parameters are used to process the rest of the data. This procedure is referred to as template processing. In this study data from the first five injections were not included in the statistical analysis, since these injections are intended to condition the column.

Procedures:

In general for GPC analyses new columns are equilibrated with mobile phase for a minimum of 24 hours. Once a column has been equilibrated flow is maintained at the analysis flow-rate throughout the life of the column where possible. This procedure also helps insure that noise in the MALS detector will be kept to a minimum. A single Lot of FERRLECIT® was used (Lot # 1T612, Exp 12 2004) for all studies described in this research report.

### EXAMPLE 1

### Reproducibility Study I

For this study, 30 injections were made from each of two ampoules of FERRLECIT® diluted 1 to 10 with mobile phase. Data from the first five injections were not included in the statistical analyses. A run-time of 45 minutes was selected, since benzyl alcohol elutes from the column at approximately 73 minutes (DRI detector), so that during each subsequent injection the benzyl alcohol would elute at a retention time of ˜28 minutes (DRI detector). The benzyl alcohol was well separated from the iron complex, which elutes at a retention time of ˜19 minutes (MALS detector) during a sequential run.

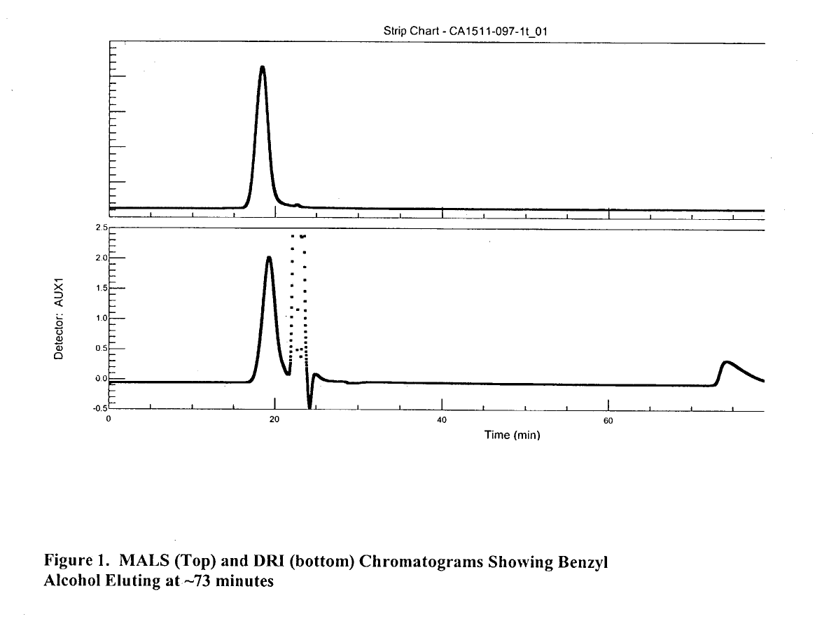

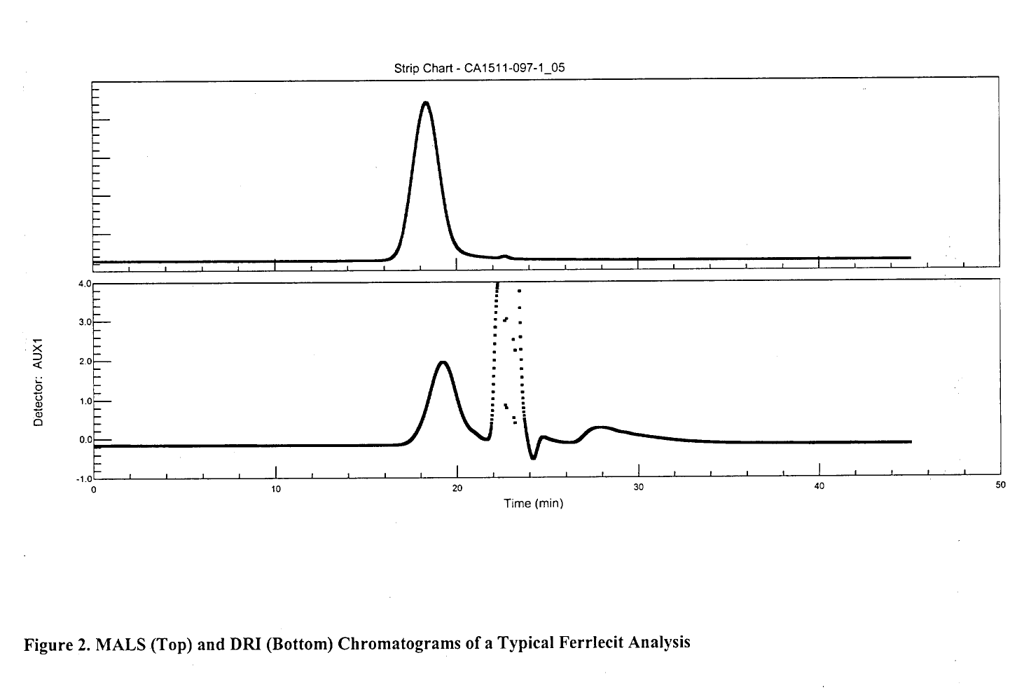

MALS and DRI chromatograms for a FERRLECIT® analysis that was allowed to run ninety minutes are shown in FIG. 1. The DRI chromatogram clearly shows benzyl alcohol eluting at ˜75 minutes. In order to shorten the analysis time and maintain column conditioning the final experimental design incorporated a run-time of 45 minutes. In each subsequent analysis, benzyl alcohol eluted at ˜28 minutes (73 min.−45 min.=28 min.). Using this experimental design, typical MALS and DRI chromatograms for a FERRLECIT® analysis are shown in FIG. 2. Benzyl alcohol can be observed eluting at ˜28 minutes in the DRI chromatogram of FIG. 2. In FIGS. 1 and 2 large off-scale peaks at ˜22-23 minutes in the DRI chromatograms result from sucrose. In the MALS chromatogram sucrose is barely visible and benzyl alcohol is not visible at all, which is a consequence of a light scattering detector's decreased sensitivity to low molecular weight molecules.

Measuring the true molecular weight, Mw, rather than either number average molecular weight (Mn) or Z average molecular weight (Mz), which are indirectly derived numbers, is preferable. Using the DRI facilitates the Mw measurement for Fe-sugar complexes because it provides a good estimate of dn/dc.

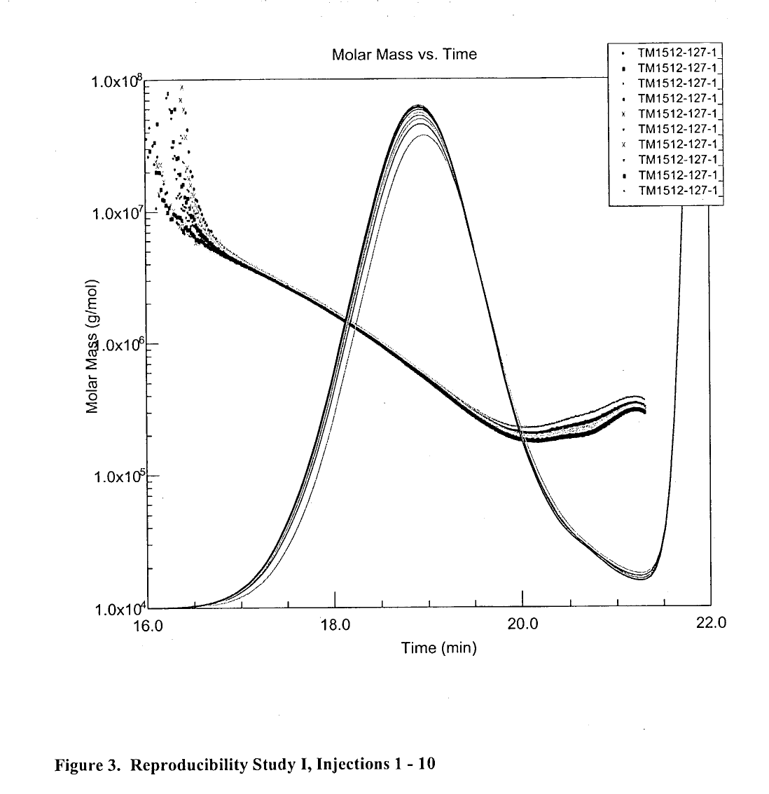

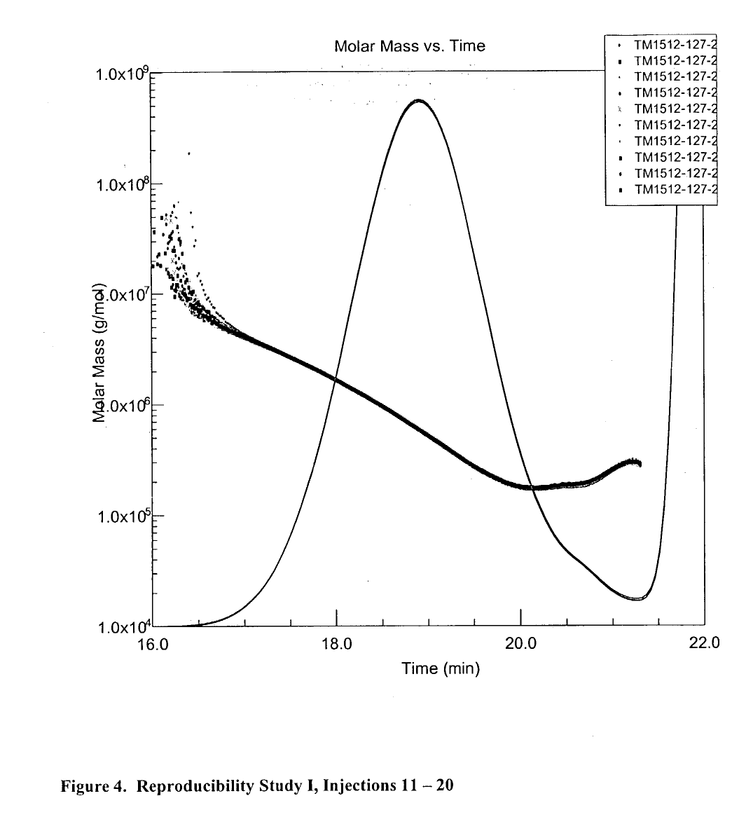

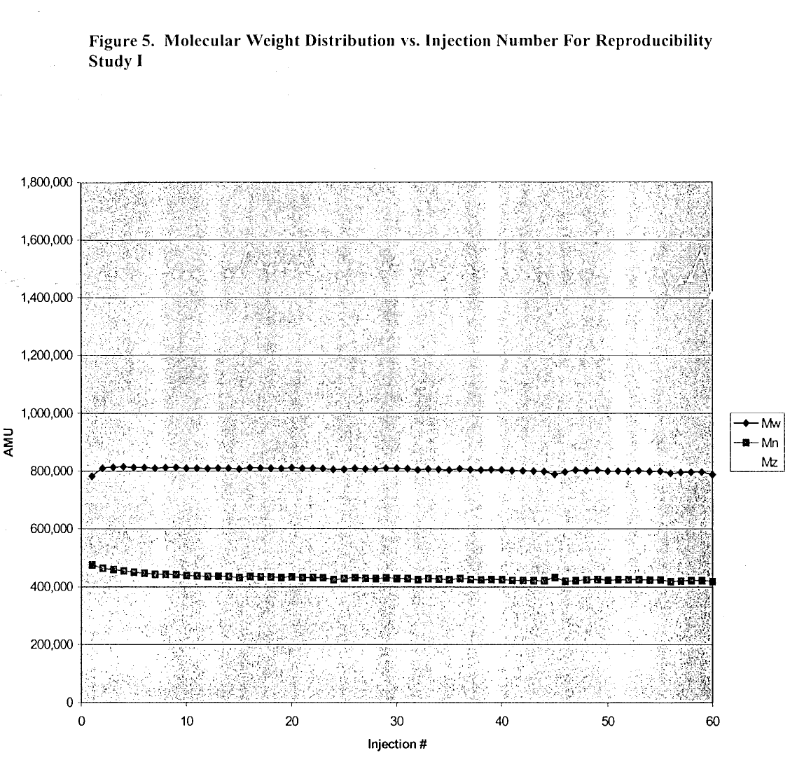

Data for the sixty injections included in Reproducibility Study I were collected. A representative portion of the data were displayed as FIGS. 3 and 4. The plots are grouped into ten injections each for the sake of simplicity with the Molar Mass vs. Time plots superimposed on the elution curves. The individual values calculated for Mn, Mw, Mz and polydispersity (Pd) for each of the sixty injections along with statistical evaluation of data (excluding first five injections) were shown in Table 1. FIG. 5 describes Molecular Weight Distribution vs. Injection Number plotted for Mn, Mw, and Mz values in Table 1.

TABLE 1
Calculation of Mn, Mw, Mz Pd and Statistical
Evaluation of Data for Reproducibility Study 1.

| | | | | Polydispersity |
|---|---|---|---|---|
| Injection # | Mn | Mw | Mz | (Pd) |
| *1 | 475,700 | 782,300 | 1,374,000 | 1.644 |
| *2 | 464,700 | 809,600 | 1,476,000 | 1.742 |
| *3 | 459,400 | 813,500 | 1,490,000 | 1.771 |
| *4 | 455,900 | 815,200 | 1,518,000 | 1.788 |
| *5 | 451,500 | 813,000 | 1,535,000 | 1.801 |
| 6 | 447,400 | 812,300 | 1,502,000 | 1.816 |
| 7 | 444,300 | 810,600 | 1,470,000 | 1.824 |
| 8 | 444,300 | 812,700 | 1,501,000 | 1.829 |
| 9 | 442,900 | 812,900 | 1,484,000 | 1.835 |
| 10 | 439,200 | 809,600 | 1,480,000 | 1.843 |
| 11 | 438,300 | 810,100 | 1,484,000 | 1.848 |
| 12 | 436,200 | 808,600 | 1,480,000 | 1.854 |
| 13 | 437,300 | 811,200 | 1,501,000 | 1.855 |
| 14 | 436,000 | 810,500 | 1,486,000 | 1.859 |
| 15 | 432,700 | 807,600 | 1,485,000 | 1.866 |
| 16 | 436,700 | 812,000 | 1,576,000 | 1.859 |
| 17 | 435,000 | 810,900 | 1,516,000 | 1.864 |
| 18 | 434,200 | 809,900 | 1,501,000 | 1.865 |
| 19 | 433,300 | 809,400 | 1,508,000 | 1.868 |
| 20 | 434,900 | 812,400 | 1,510,000 | 1.868 |
| 21 | 432,600 | 809,700 | 1,506,000 | 1.872 |
| 22 | 432,600 | 809,600 | 1,493,000 | 1.871 |
| 23 | 431,800 | 808,800 | 1,498,000 | 1.873 |
| 24 | 424,900 | 806,100 | 1,467,000 | 1.877 |
| 25 | 429,100 | 806,300 | 1,439,000 | 1.879 |
| 26 | 431,500 | 809,300 | 1,512,000 | 1.876 |
| 27 | 429,900 | 807,900 | 1,504,000 | 1.879 |
| 28 | 428,800 | 807,600 | 1,491,000 | 1.884 |
| 29 | 431,200 | 810,600 | 1,508,000 | 1.88 |
| 30 | 429,300 | 809,200 | 1,532,000 | 1.885 |
| 31 | 429,300 | 808,500 | 1,532,000 | 1.883 |
| 32 | 425,900 | 804,300 | 1,466,000 | 1.889 |
| 33 | 428,800 | 807,900 | 1,552,000 | 1.884 |
| 34 | 427,800 | 806,600 | 1,504,000 | 1.886 |
| 35 | 425,400 | 803,600 | 1,467,000 | 1.889 |
| 36 | 429,400 | 808,300 | 1,534,000 | 1.882 |
| 37 | 426,400 | 805,200 | 1,498,000 | 1.888 |
| 38 | 424,400 | 803,700 | 1,484,000 | 1.894 |
| 39 | 426,300 | 805,700 | 1,504,000 | 1.89 |
| 40 | 424,900 | 804,100 | 1,490,000 | 1.892 |
| 41 | 423,000 | 801,900 | 1,491,000 | 1.896 |
| 42 | 422,900 | 801,000 | 1,479,000 | 1.894 |
| 43 | 422,600 | 800,500 | 1,478,000 | 1.894 |
| 44 | 421,900 | 799,200 | 1,471,000 | 1.894 |
| 45 | 433,000 | 789,700 | 1,343,000 | 1.824 |
| 46 | 419,400 | 797,400 | 1,426,000 | 1.902 |
| 47 | 422,200 | 803,700 | 1,480,000 | 1.904 |
| 48 | 424,600 | 801,800 | 1,516,000 | 1.888 |
| 49 | 426,300 | 803,900 | 1,492,000 | 1.886 |
| 50 | 423,700 | 800,600 | 1,497,000 | 1.89 |
| 51 | 424,900 | 800,400 | 1,491,000 | 1.884 |
| 52 | 425,100 | 800,100 | 1,524,000 | 1.882 |
| 53 | 425,900 | 801,700 | 1,537,000 | 1.883 |
| 54 | 423,900 | 799,300 | 1,487,000 | 1.886 |
| 55 | 424,400 | 800,200 | 1,489,000 | 1.886 |
| 56 | 419,200 | 793,100 | 1,426,000 | 1.892 |
| 57 | 421,000 | 796,000 | 1,461,000 | 1.891 |
| 58 | 422,300 | 796,800 | 1,469,000 | 1.887 |
| 59 | 422,700 | 797,400 | 1,550,000 | 1.886 |
| 60 | 419,100 | 788,600 | 1,408,000 | 1.881 |
| Mean | 429,293 | 805,036 | 1,490,364 | 1.88 |
| STDEV | 6789 | 5835 | 35265 | 0.02 |
| % RSD | 1.58 | 0.72 | 2.37 | |

Sample: FERRLECIT ® 10% solution, 100 μL injection
Method: 8301aq; 100 mM NaNO₃, Shodex SB 806M SEC column
dn/dc: 0.080

The Molar Mass vs. Time plots described by FIG. 3 are characteristic of polydisperse systems and show a more or less linear decrease in molar mass as a peak elutes.

FIG. 3 clearly shows that peak heights increase slightly with each subsequent injection for the first few injections resulting in an initial increase in the calculated molecular weight. After five injections the increase becomes negligible. (Contrast this with FIG. 4). As an example, the average Mw for the first five injections is 806,720 with a % RSD of 1.7%. The average Mw for the next five injections is 811,620 with a % RSD of 0.18%, nearly 1/10 the % RSD value for the first five injections.

From the above, it is preferable that when beginning a new group of analyses, the column be conditioned. One method of conditioning would be to make about five injections prior to collecting data.

FIG. 5 (Molecular Weight Distribution vs. Injection Number) shows the reproducibility of individual molecular weight determinations for Mz, Mw, and Mn for injections 6 through 60. Referring to Table 1 Mn, Mw, and Mz demonstrate mean values of 429,293, 805,036 and 1,490,364 daltons with corresponding % RSD's of 1.58, 0.72, and 2.37, respectively, while the average Pd is 1.88 with a 1.10% RSD. The mean value for Mw ranged from about 811,620 to about 794,380 daltons. The Pd value (Mw/Mn) ranged from about 1.816 to about 1.886. In both cases, the upper and lower ends of the ranges were not statistically significant.

As was mentioned elsewhere, a measurement of radius of gyration (synonymous with rms radius) is feasible with MALS. For example, excluding the first five injections a weight averaged rms radius for the Fe-sugar complex was calculated to be 4.9±1 nm (Rw).

Based upon peak shapes and areas over the series of experiments performed in this study, there was no evidence to suggest that the sensitivity of the MALS or the DRI detectors was decreasing with either time or injection number This would be the case, for example, if some material were coating the windows of either detector cell causing a decrease in MALS and DRI outputs with increased use. On the contrary, the DRI signal increased slightly with injection number, suggesting that some low molecular weight substance might contribute to the small change (DRI is more sensitive to low molecular weight compounds than high molecular weight substances). In addition if the window of MALS cell were being coated, background interference would increase dramatically due to spurious light scattering. Further, intermittent in situ ultrasonic cleaning of the MALS cell does not increase its output.

### EXAMPLE 2

### Grouped Reproducibility Study

In the second study eight injections were made in five groups with a 2-hour equilibration period between the first four groups and five hours between the fourth and fifth group. The injection sample was prepared from a newly opened FERRLECIT® ampoule diluted 1 to 10. As above, the data from the first five injections in each group were not processed.

The Grouped Reproducibility Study was designed to give some idea of how the period of time in between injections affects the column conditioning in a series of analyses. More specifically, this study was designed to determine how much equilibration time in between injections constitutes a new series.

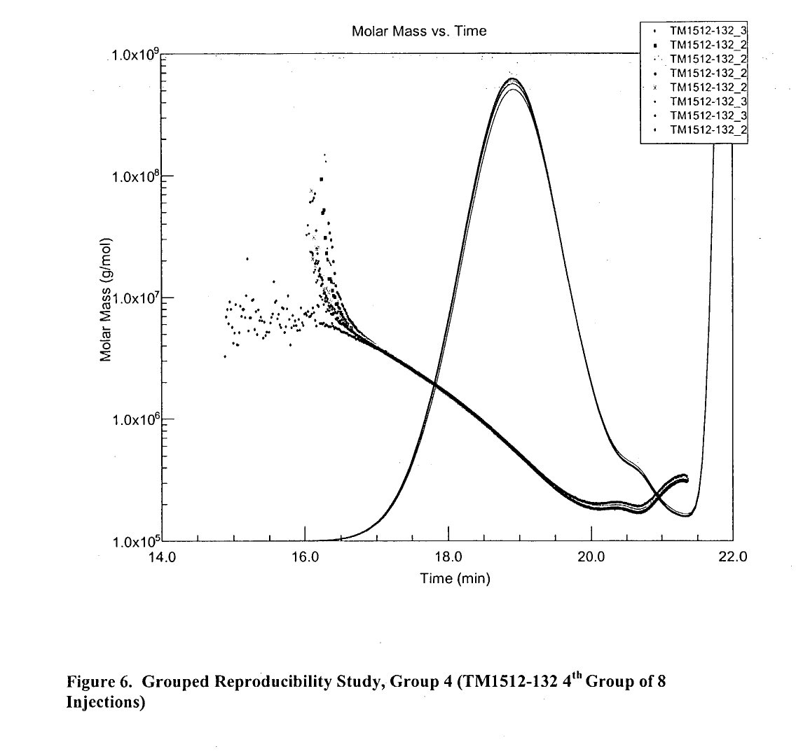

A two-hour equilibration time was allowed between Groups 1 and 2, 2 and 3, 3 and 4 and five hours between Groups 4 and 5. FIG. 6 is a typical plot, exemplifying Group 4 data for Molar Mass vs. Time, along with the corresponding elution curves. Table 2 shows calculated values of Mn, Mw, Mz, and Pd for individual injections for Group 4.

TABLE 2
Calculation of Mn, Mw, Mz Pd and Statistical
Evaluation of Data in Grouped Reproducibility
Study for Group 4.

| | | | | Polydispersity |
|---|---|---|---|---|
| Injection # | Mn | Mw | Mz | (Pd) |
| 1 | 443,000 | 773,100 | 1,391,000 | 1.745 |
| 2 | 432,400 | 774,000 | 1,430,000 | 1.79 |
| 3 | 428,000 | 771,800 | 1,450,000 | 1.803 |
| 4 | 426,500 | 772,600 | 1,436,000 | 1.811 |
| 5 | 426,200 | 773,200 | 1,416,000 | 1.814 |
| 6 | 425,900 | 773,200 | 1,411,000 | 1.815 |
| 7 | 423,300 | 771,200 | 1,389,000 | 1.822 |
| 8 | 423,600 | 772,100 | 1,424,000 | 1.823 |
| Mean | 424,267 | 772,167 | 1,408,000 | 1.82 |
| STDEV | 1422 | 1002 | 17692 | 0.003 |
| % RSD | 0.34 | 0.13 | 1.26 | 0.24 |

Sample: FERRLECIT ® 10% solution, 100 μL injection
Method: 8301aq; 100 mM NaNO₃, Shodex SB 806M SEC column
dn/dc: 0.080

As in FIG. 3, the peak heights increase with each subsequent injection for the first few injections resulting in an initial increase in the calculated molecular weight. After five injections the increase becomes negligible.

The means for Mw ranged for Groups 2, 3, and 4 from about 775,100, to 773,267 and to 772,167 daltons, respectively. The equilibration time between these groups was two hours. No decrease in mean Mw was observed between Groups 4 and 5 in which a five-hour equilibration time was allowed. The calculated Rw based upon an average of the last three injections in each group for all five groups is 5.8±2 nm.

Column conditioning subsists for less than two hours, since re-conditioning may be required when injections are delayed by this period of time. As a matter of protocol within a given group of analyses if the column is allowed to equilibrate for the time required for one run (45 minutes), it is preferable that the column be re-conditioned by making about five injections prior to collecting new data (three injections). It can also be concluded based on this study that between different groups of analyses, equilibration should be allowed to proceed, preferably, about two hours. For example, when analyzing three separate Lots of FERRLECIT® each sample may be injected eight times, but only the average and % RSD may be calculated for the last three injections. The column may then be equilibrated two hours and the second Lot processed as above. After a two-hour equilibration period the third Lot may be analyzed.

### EXAMPLE 3

### Column Cleaning

On completion of the Grouped Study the column was equilibrated overnight and cleaned with 100 mM EDTA adjusted to pH 5.0 with acetic acid containing 200 ppm sodium azide for 48 hours. The column was equilibrated overnight with mobile phase prior to initiation of Reproducibility Study II.

### EXAMPLE 4

### Reproducibility Study II

After re-equilibration, Reproducibility Study II was conducted in exactly the same fashion as the sixty-sample study; however, 25 injections from each of two samples were made for a total of fifty injections.

Prior to performing the second reproducibility study, the system was treated for 48 hours with 100 mM EDTA solution adjusted to pH 5 to remove Fe-sugar complexes that might have adhered to stationary phase.

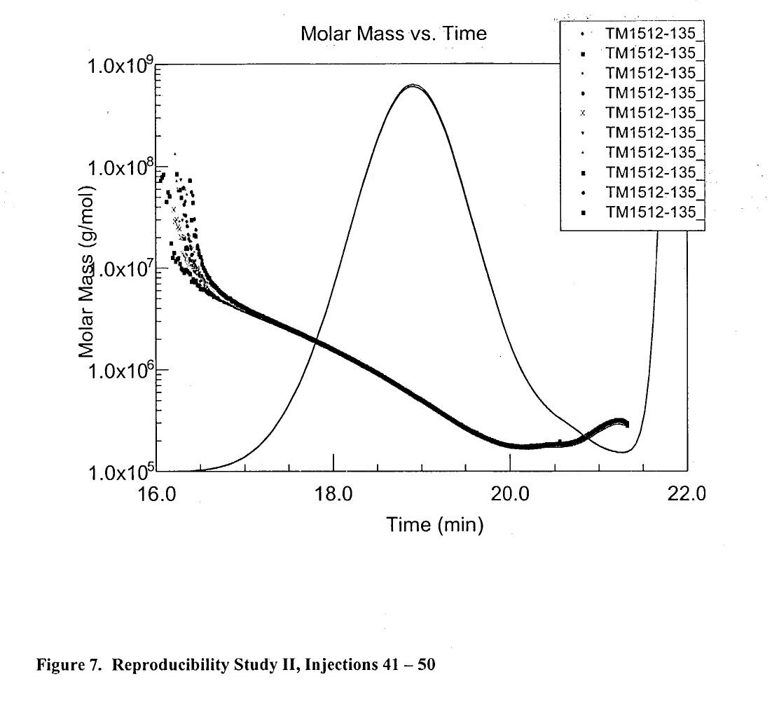

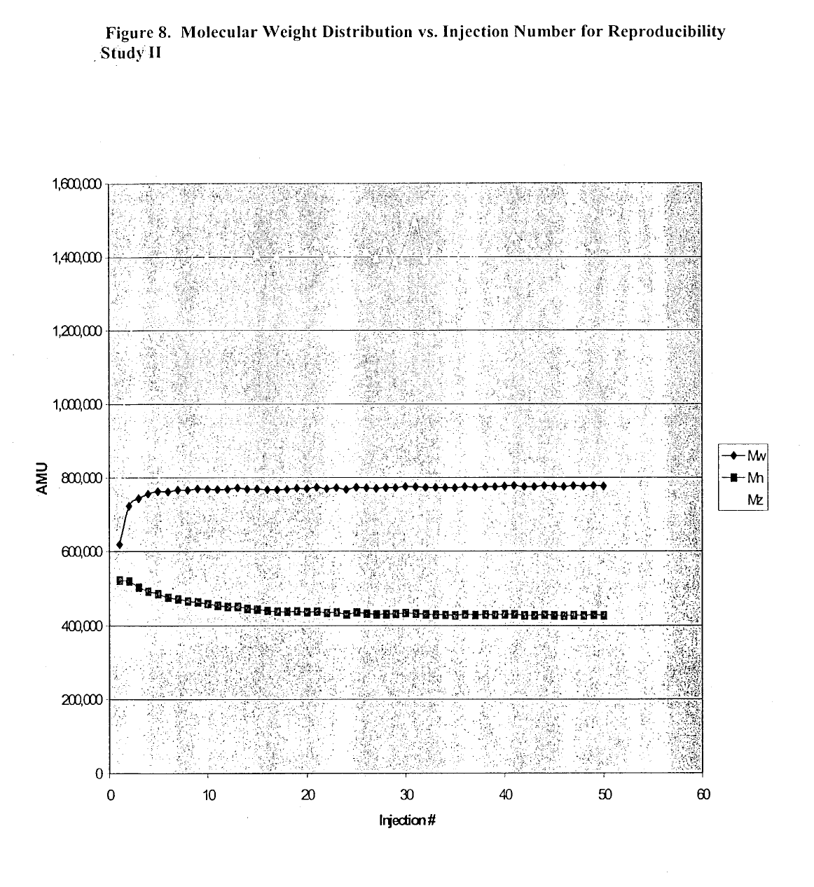

FIG. 7 displays a typical plot for the Data for the Reproducibility Study II. The study was grouped into ten injections each with the Molar Mass vs. Time plots superimposed on the elution curves. The individual values calculated for Mn, Mw, Mz and polydispersity (Pd) for each of the fifty injections along with statistical evaluation of data (excluding first five injections) are shown in Table 3. FIG. 8 displays Molecular Weight Distribution vs. Injection Number plotted for Mn, Mw, and Mz values in Table 3.

TABLE 3
Calculation of Mn, Mw, Mz Pd and Statistical Evaluation
of Data for Reproducibility Study II.

| | | | | Polydispersity |
|---|---|---|---|---|
| Injection # | Mn | Mw | Mz | (Pd) |
| *1 | 522,300 | 619,000 | 798,500 | 1.185 |
| *2 | 520,000 | 723,600 | 1,170,000 | 1.391 |
| *3 | 503,200 | 743,900 | 1,241,000 | 1.478 |
| *4 | 492,600 | 756,000 | 1,329,000 | 1.535 |
| *5 | 485,700 | 763,100 | 1,355,000 | 1.571 |
| 6 | 475,700 | 762,000 | 1,349,000 | 1.602 |
| 7 | 471,800 | 766,800 | 1,383,000 | 1.625 |
| 8 | 465,700 | 766,300 | 1,374,000 | 1.646 |
| 9 | 463,400 | 769,700 | 1,419,000 | 1.661 |
| 10 | 458,600 | 768,700 | 1,397,000 | 1.676 |
| 11 | 454,400 | 768,000 | 1,372,000 | 1.69 |
| 12 | 451,000 | 768,200 | 1,371,000 | 1.704 |
| 13 | 450,900 | 772,200 | 1,433,000 | 1.713 |
| 14 | 445,400 | 768,800 | 1,452,000 | 1.726 |
| 15 | 443,500 | 769,200 | 1,410,000 | 1.734 |
| 16 | 439,900 | 767,500 | 1,376,000 | 1.745 |
| 17 | 437,600 | 767,200 | 1,402,000 | 1.753 |
| 18 | 437,500 | 768,400 | 1,415,000 | 1.757 |
| 19 | 438,600 | 771,500 | 1,429,000 | 1.759 |
| 20 | 436,300 | 770,200 | 1,413,000 | 1.766 |
| 21 | 437,700 | 773,700 | 1,442,000 | 1.768 |
| 22 | 434,200 | 769,900 | 1,402,000 | 1.773 |
| 23 | 435,600 | 772,600 | 1,471,000 | 1.773 |
| 24 | 430,500 | 768,000 | 1,374,000 | 1.784 |
| 25 | 435,500 | 773,800 | 1,418,000 | 1.777 |
| 26 | 432,100 | 772,500 | 1,415,000 | 1.788 |
| 27 | 430,400 | 770,600 | 1,397,000 | 1.79 |
| 28 | 430,800 | 772,600 | 1,441,000 | 1.793 |
| 29 | 431,700 | 772,700 | 1,389,000 | 1.79 |
| 30 | 433,800 | 775,900 | 1,415,000 | 1.788 |
| 31 | 432,100 | 775,300 | 1,495,000 | 1.794 |
| 32 | 430,600 | 773,100 | 1,394,000 | 1.796 |
| 33 | 429,600 | 773,400 | 1,430,000 | 1.80 |
| 34 | 429,000 | 772,800 | 1,415,000 | 1.801 |
| 35 | 427,500 | 772,200 | 1,384,000 | 1.806 |
| 36 | 429,900 | 775,000 | 1,426,000 | 1.803 |
| 37 | 428,400 | 773,400 | 1,391,000 | 1.805 |
| 38 | 429,400 | 775,300 | 1,437,000 | 1.805 |
| 39 | 428,800 | 775,000 | 1,417,000 | 1.807 |
| 40 | 430,200 | 776,800 | 1,414,000 | 1.806 |
| 41 | 430,100 | 778,300 | 1,457,000 | 1.809 |
| 42 | 427,400 | 775,400 | 1,435,000 | 1.814 |
| 43 | 427,600 | 775,400 | 1,438,000 | 1.813 |
| 44 | 429,100 | 777,400 | 1,454,000 | 1.812 |
| 45 | 427,600 | 776,100 | 1,416,000 | 1.815 |
| 46 | 426,700 | 775,700 | 1,431,000 | 1.818 |
| 47 | 427,900 | 777,500 | 1,441,000 | 1.817 |
| 48 | 426,900 | 776,200 | 1,423,000 | 1.818 |
| 49 | 428,400 | 777,900 | 1,432,000 | 1.816 |
| 50 | 427,000 | 776,500 | 1,432,000 | 1.819 |
| Mean | 437,262 | 772,349 | 1,416,022 | 1.768 |
| STDEV | 12723 | 3747 | 29057 | 0.056 |
| % RSD | 2.91 | 0.49 | 2.05 | 3.15 |

Sample: FERRLECIT ® 10% solution, 100 μL injection
Method: 8301aq; 100 mM NaNO₃, Shodex SB 806M SEC column
dn/dc: 0.080

A comparison of data of Table 3 showing the first ten injections from the current reproducibility study with FIG. 3, which shows the first ten injections for Reproducibility Study I, indicates that there is more variability in the first five injections in the current study. For example the mean of the first five injections in the present study is 721,120 daltons with an 8.18% RSD compared to 806,720 with a % RSD of 1.71 for the first reproducibility study. The mean of the next five injections in this study (Injection 6-10) is 766,700 daltons with only a 0.38% % RSD. “Cleaning” of the system with acidic EDTA, may introduce more variability in the early injections, perhaps by opening up “active sites” on the stationary phase. Even though there is more variability in data from the first few injections, when the data are considered as a whole (excluding Injections 1-5), intra-study reproducibility is good with a mean Mw of 772,349 daltons and a % RSD of 0.49%.

The downward trend in Mw in Reproducibility Study I and the Grouped Reproducibility study appears to have stopped or even reversed by cleaning (see FIG. 8). Without wishing to be bound to any particular theory, it is hypothesized that this result may be attributable to the effect of cleaning with acidic EDTA. Thus, it may be preferable to clean the column for twenty-four hours with acidic EDTA prior to equilibration with mobile phase. In addition, it may be preferable to clean the column after an arbitrary number of injections, for example one hundred.

### EXAMPLE 5

### Response Study

Finally, a response study was conducted immediately following Reproducibility Study II in which three injections each of 100, 90, 80, 70, 60, and 50 μL of diluted FERRLECIT® (1:10) were made from a new ampoule of FERRLECIT®. Since the samples in the response study were from the same Lot of FERRLECIT® and at the same concentration, no equilibration was allowed between Reproducibility Study II and the Response Study.

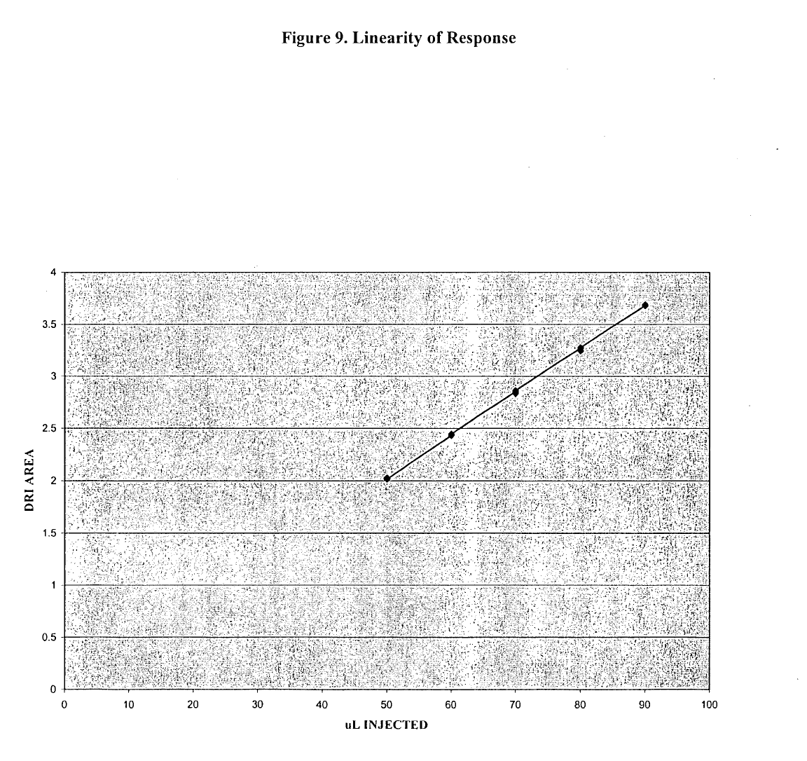

Linearity of response data for the DRI detector as a function of injected amount along with statistical evaluation of data are summarized in Table 4 below. The data are displayed in FIG. 9 which shows excellent correlation between the peak area of the DRI detector and the quantity of sample injected over the range of 50 μL to 100 μL injections. In addition in order to see if injection volume affected molecular weight calculations, Mw was calculated for each injection (Table 4). Injection Volume has no effect on the calculation. When data from this study were used to calculate the radius of gyration, Rw=2.9±2 nm.

TABLE 4
Linearity of Response Data for the
DRI Detector (Mw Calculated From Data)

| Injection Volume | DRI Area | Area/Volume | Mw |
|---|---|---|---|
| 50 | 2.02761 | 0.04055 | 783,600 |
| 50 | 2.01818 | 0.04036 | 785,300 |
| 50 | 2.01613 | 0.04032 | 787,100 |
| 60 | 2.43178 | 0.04053 | 784,700 |
| 60 | 2.43004 | 0.0405 | 783,600 |
| 60 | 2.44638 | 0.04077 | 780,700 |
| 70 | 2.85044 | 0.04072 | 781,300 |
| 70 | 2.83263 | 0.04047 | 782,900 |
| 70 | 2.86278 | 0.0409 | 779,600 |
| 80 | 3.27165 | 0.0409 | 780,000 |
| 80 | 3.24555 | 0.04057 | 782,000 |
| 80 | 3.27672 | 0.04096 | 777,500 |
| 90 | 3.68235 | 0.04092 | 778,400 |
| 90 | 3.67951 | 0.04088 | 779,500 |
| 90 | 3.68978 | 0.041 | 778,000 |
| | Mean | 0.04069 | 781,600 |
| | STDEV | 0.00023 | 2870 |
| | % RSD | 0.56297 | 0.37 |

Sample: FERRLECIT ® 10% solution, 100 μL injection
Method: 8301aq; 100 mM NaNO₃, Shodex SB 806M SEC column
dn/dc: 0.080

Weight Average Molecular Weight and Radius of Gyration:

Based on about 130 data points collected in this study, the radius of gyration (Rw) is determined to be 4.4±1.8 nm and the mean weight average molecular weight for the Fe-sugar complex in FERRLECIT® is 787,500±15,200 daltons. Accuracy of these values can be improved further by refining the value for specific refractive increment, dn/dc.

While it is apparent that the embodiments of the invention herein disclosed are well suited to fulfill the objectives stated above, it will be appreciated that numerous modifications and other embodiments may be implemented by those skilled in the art, and it is intended that the appended claims cover all such modifications and embodiments that fall within the true spirit and scope of the present invention.

What is claimed is:

1. A method of assaying a Fe-Sugar complex from an injectable pharmaceutical composition comprising:
a) using a polyhydroxymethacrylate column coupled with a multiangle light scattering detector operating at a wavelength of about 600 to about 800 nm;
b) placing a suitably prepared sample of said injectable composition onto said column;
c) using an aqueous eluent comprising an alkali-nitrate;
d) estimating specific refractive increment (dn/dc); and
e) calculating true molecular weight (Mw), number average molecular weight (Mn), Z-average molecular weight (Mz), polydispersity (Pd) or a combination thereof.

2. The method according to claim 1, for assuring the quality of an injectable pharmaceutical composition that contains a Fe-Sugar complex, further comprising:
rejecting said composition for distribution into stream of commerce if any of said true molecular weight, number average molecular weight, Z-average molecular weight, polydispersity or a combination thereof falls outside a pre-selected range.

3. The method according to claim 1 or 2, wherein said detector operates at about 690 nm and said alkali nitrate is sodium nitrate at about 100 mM concentration.

4. A Fe-sugar complex containing pharmaceutical composition having a molecular weight between about 750,000 and about 820,000 as determined by a chromatographic assay, comprising:
a) using a polyhydroxymethacrylate column coupled with a multiangle light scattering detector operating at a wavelength of about 600 to about 800 nm;
b) placing a suitably prepared sample of said injectable composition onto said column;
c) using an aqueous eluent comprising an alkali-nitrate;
d) estimating specific refractive increment (dn/dc); and
e) calculating true molecular weight (Mw), number average molecular weight (Mn), Z-average molecular weight (Mz), polydispersity (Pd) or a combination thereof.

5. The Fe-sugar complex according to claim 4, wherein said detector operates at about 690 nm and said alkali nitrate is sodium nitrate at about 100 mM concentration.
